(19) United States
(12) Patent Application Publication
(10) Pub. No.: US 2020/0188577 A1
(43) Pub. Date: Jun. 18, 2020

(54) MEDICAL PORT LOCATOR SYSTEM AND METHOD

(71) Applicant: MARK SIPE, PHOENIX, AZ (US)

(72) Inventor: MARK SIPE, PHOENIX, AZ (US)

(21) Appl. No.: 16/221,004

(22) Filed: Dec. 14, 2018

(51) Int. Cl. A61M 5/142; A61M 39/02; A61M 5/42

(52) U.S. Cl. CPC A61M 5/14244; A61M 39/0208; A61M 5/425; A61M 2005/14284; A61M 2039/0232; A61M 5/427

(57) ABSTRACT

A needle guide is used to locate a corresponding implanted medical port. The needle guide automatically centers over the embedded or body surface directly over the implanted device via magnetism. Finding and accessing into the catheter port is made quickly and accurately, effectively eliminating the need for multiple shot attempts and excessive touching of the skin around the implant.

### TECHNICAL FIELD

This invention relates to a system and method for medical ports, and more specifically the invention provides a locatable port-a-cath medical port system and method for locating implantable devices through a patient's skin.

### RELATED ART

Many individuals experience health issues that may require multiple medical shots, saline or other cleansing flushes, or infusions over a course of treatment. Infusions of pharmaceutical drugs may be administered, and/or blood samples may be drawn by hypodermic needles, however the repeated need to access the patient's vascular system can lead to bruising and difficulty to find appropriate treatment sites. Therefore, medical ports have beat designed as an implantable device to provide access to a patient's vascular system, establish a single location for hypodermal intrusion, and may include a catheter with direct access to a vein or artery. A port (commonly an implantable venous access device) is a small medical appliance that may be installed beneath the skin of a patient. A catheter may provide fluid connection from the port to a vein. The port may include a septum (typically made of a self-sealing compressed silicone) through which a hypodermic needle may access a reservoir wherein drugs may be injected (and/or blood samples/fluids can be drawn).

There is a risk involved with implantable ports for infections to be caused if not properly maintained. Oftentimes, ports are installed in the upper chest below the clavicle or collar bone. Depending on the patient, the thickness of the dermis, or other factors, it may be difficult to locate the port and septum to provide accurate access via a hypodermic needle. A suitable solution for easily and reliably locating an injection point on ports is desirable.

Earlier advances in access ports have included light emitters for indicating the location of the port. Prior art light emitters have been powered by coils and capacitors, etc. However, there has yet to be a complete system with an indicator of location, a matching mating device, and activator within the device.

It is therefore an object of the present invention to provide a mechanical location assistance for a medical port accessible from outside of a patient's body.

It is a further object of the present invention to assist caregivers to locate an implantable device.

It is as yet a further object of the present invention to provide a method for ease of use in locating an implantable device.

### SUMMARY OF THE INVENTION

The present invention provides a reliable and efficient means by which to locate an implantable device, such as a medical port, to assist in care.

A medical tool system for verifying proper placement of a medical instrument with respect to an implantable medical device is disclosed herein. The medical system includes a port, preferably already implanted below the skin, as is known in the art. The port may include a reservoir adapted to store fluid, and an implantable housing. The housing may define a port through which fluid access to the reservoir is obtained. The housing preferably includes an upper rim disposed towards the underside of a patient's skin (when installed), and an aperture (often along the side or bottom) adapted to provide fluid communication with a portion of the patient's vascular system (often through tubing or catheter sealed onto a portion of the housing).

The housing may include one or more, preferably at least two magnetically responsive material (MRM) (such as magnets, metals, mineral material, or other magnetically responsive materials known in the art or as may be found to be useful in the present device to allow magnetic coupling of a handle device and an implanted device), preferably in pins or blocks. The MRM may be along or inside the housing, on side walls, or around the rim, or more preferably around the reservoir within the housing. Preferably the MRM will be arranged, or arrayed, in multiple geometric points around the reservoir. Two MRM may be arranged on either side of the reservoir. The two port-based MRM may include reverse polarity relative one another (flipped), and arranged to mate with complementary magnets or MRM in the locator tool. Alternatively, three or more MRM may be arranged separately (and possibly flipped) in the port to coincide with complementary MRM in the locator tool. Alternatively, the MRM arranged in the port may all have the same polarity direction to match with a ring MRM in the locator tool. Similarly, the port may have a ring MRM to mate with a ring in the locator tool, or two or more MRM in the locator tool. In some cases, the port-based MRM are placed over the rim, and others, the magnets are integral with the rim or in the housing. In some embodiments, the MRM may be a single circular (or a portion thereof) that may sit atop, along, within, or in place of the port rim, or in some cases along different points of perimeter of housing. The MRM is adapted to provide one or multiple points of attraction that can respond to, modify, or be attractive, magnetic field(s) beyond the patient's body (through the skin).

A locator tool magnet may be adapted to mate with a MRM on, or in, the port within the patient's body (magnetic field produced by a one or more magnets in/on the port, in/on the handle, both, or in response to, or as modifying a prepared magnetic field). The MRM may be part of a handheld locator, along a bottom side, in a needle guide (such as a ring magnet) or otherwise. The locator tool MRM may be arranged with complementary polarity to match with port-based MRM.

Certain embodiments may comprise illumination means for help in reliably locating the point of insertion for a needle or other medical tool to access the reservoir and/or vascular system, or otherwise. The light may be emitted through the skin to help identify housing features, such as rim, septum, etc. The light may be powered by a source under the skin, within the body, or remotely from outside the body. A thermoelectric generator may be employed to power a battery that can store and selectively release power to light (and other electronic devices in the system).

A method of supplying a medication, or otherwise accessing fluids in a patient, is also disclosed herein. A medical port with at least a second MRM is implanted under a film or under the skin. A locator tool with a first MRM is applied over the film, outside the body, to mate with MRM provided by the second embedded MRM associated with the medical port. The external locator tool may activate a Reed switch, or otherwise close a circuit, or otherwise activate a light. A caregiver may insert a medical instrument, such as a hypodermic needle to supply medication, into the medical port, preferably through an aperture provided in a handheld instrument, and then through the film/skin. The method may further include activating a light coupled to the medical port with the external locator magnet (or magnetically responsive material), or other feature in the handheld, or by pressure on the skin. The light may be powered by a battery that is charged via a thermoelectric generator or other suitable means. The power source, may also include a capacitor that is charged by a battery, thermoelectric generator or otherwise.

### BRIEF DESCRIPTION OF THE DRAWINGS

FIG. 1 illustrates a side sectional view of a prior art medical port system.

FIG. 2 illustrates a side sectional view of an installed medical port system with hand locator tool.

FIG. 3 is a top perspective view of a locator tool of an embodiment of the present invention.

FIG. 4A is a top view of an implantable medical port system of an embodiment of the present invention.

FIG. 4B is a front view of an implantable medical port system of an embodiment of the present invention

FIG. 4C is a side view of an implantable medical port system of an embodiment of the present invention

FIG. 5 is a top perspective view of the implantable medical device of an embodiment of the present disclosure.

FIG. 6A is a reverse perspective sectioned view of the implantable medical device of an embodiment shown in FIG. 5 along line 6-6.

FIG 6B is a side sectioned view of the implantable medical device of an embodiment shown in FIG. 5 along line 6-6.

FIG. 7A is a side sectioned view of an implantable medical device of an embodiment.

FIG. 7B is a perspective sectioned view of an implantable medical device of an embodiment.

FIG. 8 is a flow diagram illustrating a method of use according to an embodiment of the present invention.

FIG. 9A illustrates a side view of a Reed Switch in an ON Position, without modification of a magnetic field.

FIG. 9B illustrates a side view of a Reed Switch in an ON Position, with modification of a magnetic field.

FIG. 10 illustrates a top view of an embodiment of the present invention using multiple MRMs along the rim of an implantable medical device.

FIG. 11 illustrates a top view of an embodiment of the present invention using a single ring MRM along the rim of an implantable medical device.

FIG. 12 illustrates a side sectional view of a needle passing through a layer of skin and into an implantable medical device of an embodiment of the present.

FIG. 13 illustrates a side sectional view of a needle passing through a layer of skin and into an implantable medical device of an embodiment to communicate with a vascular system.

FIG. 14 illustrates a top view of a locator tool of an embodiment of the present invention.

FIG. 15 illustrates a side cross-sectional view of the locator tool of FIG. 14 along line 15-15.

FIG. 16 illustrates a side view of a locator tool of the embodiment shown in FIG. 14.

FIG. 17 illustrates a bottom view of a locator tool of the embodiment shown in FIG. 14.

FIG. 18 illustrates a partial top perspective view of a locator tool of the embodiment shown in FIG. 14.

FIG. 19 illustrates a front view of a locator tool of the embodiment shown in FIG. 14.

FIG. 20 illustrates a top perspective view of a locator tool of the embodiment shown in FIG. 14.

FIG. 21 illustrates a top view of a locator tool of an embodiment of the present invention.

FIG. 22 illustrates a side view of a locator tool of the embodiment shown in FIG. 21.

FIG. 23 illustrates a bottom view of a locator tool of the embodiment shown in FIG. 21.

FIG. 24 illustrates a top perspective view of a locator tool of the embodiment shown in FIG. 21.

### DETAILED DESCRIPTION

A guide, or handheld locator tool, is used to assist in the location of a corresponding device, such as a medical port, in a human or animal or body surface. The system includes a pair of magnetic (or magnetically responsive) systems to allow a) the handheld device, with needle guide, to snap into place, and align, over b) an implanted port. The dual MRM system allows the locator tool to automatically center the guide over the embedded port device. The number and arrangement of MRM in the matching devices allows the locator tool to snap into place, and not fit tangentially along the port. By including more than one MRM in the port (and/or locator tool) along a single plane, the requirement that all MRM are engaged ensures the needle guide is directly above the septum in the port.

The handheld device preferably includes a guide as an open aperture. The locator tool needle guide includes an aperture with a diameter, or inner frame, sized slightly smaller than the interior parameter (diameter) of the housing rim of the implanted device. The aperture may be beveled (wider top, narrower bottom) to direct a needle towards the center, and ensure proper use of the port without missing the port. This facilitates needle prick, and avoids needle tip touching aspects of the port that may be damaged (or damage the needle tip) such as housing rim, interior walls, etc. The handheld locator tool device is used to find, center over, and facilitate injecting into port to access the catheter port, effectively eliminating misses and/or the need for multiple shot attempts. The present invention includes a magnetically responsive port design, with one, two, three, (or more) magnetically responsive materials and/or a magnetic ring, around/within/upon the edge of the port that mates with a medical tool hand-held above the patient's skin. The term “magnetically responsive material” (“MRM”) herein generally refers to materials known in the art or as may be found to be useful in the present device to allow magnetic coupling of a handheld device and an implanted device (such as magnets, metals, mineral material, or other magnetically responsive materials, etc.).

Preferably, the handheld includes a magnet that may trip a Reed switch (or suitable equivalent) within or electronically coupled to the port, to complete a circuit and/or activate a light (illuminating means) within or near the imbedded port. A Reed switch, as referred to herein, comprises an electrical switch operated by an applied magnetic field consisting of a pair of contacts. The contacts may be normally open, closing when a magnetic field is present or absent (preferred), or alternately normally closed and opening when a magnetic field is applied. The switch may be actuated and once the magnet is pulled away from the switch, the Real switch will return to its resting ‘normal’ position. Magnets may or may not have reversed polarity, and certain embodiments may have a hole in their center. Ring magnets may be employed, with openings that can flare in and down or be ‘straight’ on the inside to serve as needle guides. The Reed switch may act as a proximity switch. The light may be powered by a battery which is preferably charged via thermo-electric generator under the skin (other powering means may be employed). Alternately, mechanical generation and/or a remote charger may be used to power the battery and the light. The powering system is not required to be next to (proximate) the battery. The light may vary from embodiment to embodiment in positioning.

Certain embodiments may include a handheld device with a lower cover (such as felt, silicone, etc.) to prevent direct contact of the handheld device (with magnet/metal) on the patient's skin. Any thin coating may be used so as to avoid exposure of cold metal, plastic, or sharp surfaces in the handheld device directly to the skin so as to create an improved experience of heightened comfort for the patient.

In some embodiments, the port will be made with a magnetically responsive metal that does not innately create a (strong) magnetic field, which can react to a magnetic field and mate with a magnet in the handheld device. Preferably the handheld device includes at least one magnet(s), preferably a ring or mostly closed circle to better orient, and snap, the handheld in place over the port opening. The embedded port may include one, or preferably at least two, complementary magnets.

Referring again now to the magnets and MRM, when the port includes magnets or MRM, a plurality of magnets or MRM may be placed around the septum, either along, below, embedded, above the rim of the housing, directly below the rim, or within the housing. The magnets (or MRM) may be placed below the septum and reservoir, but may be placed upon the housing above the septum (outside the reservoir) or along the housing. In some instances the magnet or responsive material, may be within the walls of the housing under the septum (adjacent the reservoir), but isolated from the reservoir. In a preferred embodiment, the housing includes a specific profile shape with corners to accommodate locator pins or blocks with MRMs. The MRMs may be arrayed or arranged in the housing along the sides of the reservoir.

The septum preferably includes those materials known in the art, such as a self-sealing silicone which can be punctured hundreds of times before it is significantly weakened. The health care giver first locates the port (via ‘magnetic pull’), and may remove the handheld to then disinfect the area. The care giver places the handheld over the port, locating an aperture in the handheld over the septum. The care giver may then access the port by puncturing the overlying skin with a needle, or other such suitable medical tool, through the aperture in the handheld, through the skin, through the septum, and into a reservoir to create fluid access to the patient's vascular system. With certain embodiments of the locator tool (with channels), one may place or tape tubing, such as an intravenous line, while the locator tool is in place, and allow the tool to be removed thereafter.

The medical tool includes a handheld device that may be manipulated. The tool may include a handle plate and handle sides. A MRM will be appurtenant to the handheld device to interact with the embedded port. The handheld may include a single magnetic ring for optimal needle placement upon injection into and through the septum. Alternate embodiments may include a partial moon shaped magnet, a C-shaped magnet, or at least two magnets, in the handheld. These alternative shapes of MRM in the locator tool may interact with at least two separate points along the rim of the embedded port. The aperture, or preferably a center opening, of the handheld device locator tool may be slightly smaller than the septum surface to effectively ensure the needle does not catch on the inner septum ring material. A single ring of metal/magnets or multiple pieces of metal/magnets may be used as a series of triggers using both negative and/or positive polarity to initiate the opening and closing of one or multiple Reed switches or valves.

The handheld tool preferably includes a magnet, embedded in plastic to avoid damage or dislocation of the magnet. When the handheld, and preferably onboard magnet, is placed over the medical port, a Reed switch, valve, or device may be “turned on/off or opened/closed” activating the circuit or action. This may trigger a light, a piezo device, release a dose of medicine, or any number of other actions caused by (associated with) activating/deactivating a device. Using multiple control units like a Reed switch, micro-valves or magnets of different polarities is envisioned. Use of the device may also activate a string of conditions acting as a fail-safe before a final action, such as releasing medicine or initiating an action(s). The feature of using a magnet to activate a device allows a battery coupled to the port to supply power to onboard-port electronic devices to remain dormant (or charging) until the magnet is in place, thus reserving useful battery life. The Reed switch may cycle between open and closed conditions. When a magnet is placed over the port (in-use), the Reed switch is caused to close and the circuit is completed. The Reed switch is in a ‘normally-open position’ until it senses a magnet (magnetic field) in local proximity. This feature may also be used to trigger a dose of medicine, power a piezo device or other such unit. Catheter shields and suture plugs may or may not be used. Suture holes may be located on the outside base of the portal body to suture the port to the underlying tissue intra-operatively.

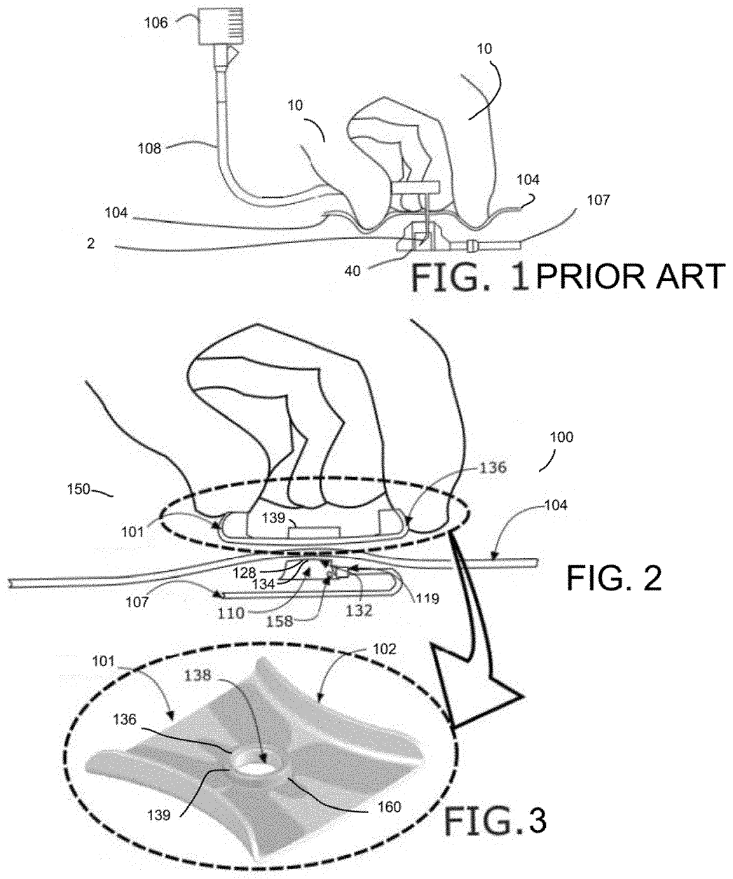

Referring now to the figures. FIG. 1 is a side sectional view of the prior art systems for utilizing a medical port using a Huber Needle 2. Medication may be provided into the patient from a syringe 106 (or fluid source, as may be known in the art) through tubing 108 as is known in the art for Huber needles. The needle 3 is provided into the port 40 through the skin 104. Port 40 typically includes catheter 107 that will connect to the vascular system. As seen, the deficiency of the prior art includes the use of fingers 10 to pinch over the port, so as to locate the port under the skin, to provide access via needle 2.

FIG. 2 illustrates a side view of an embodiment of the medical system 100 of the present invention in-use condition 150. Locator tool 101 is placed over skin 104 directly above, or in coupling contact with the implanted medical device 110. Locator tool 101 provides needle guide 136 with central aperture 138 positioned above septum 128. Port device 110 may include a second lower magnet 158 that can interact with a magnetic field of magnet 160 within locator tool 101. Locator tool 101 may be equipped with handles 102, preferably along the outside perimeter, to provide for gripping by fingers 10. Optionally, and as shown in FIG. 15, locator tool 101 may include a soft, flat material 114 along the underside 116 of tool to interface with the skin. Medical device 110 is preferably used as port, and may include an upper rim 132 that defines an aperture 134 covered by septum 128 to provide access to reservoir 120. Medical device 110 provides a port to access a catheter 107 which may be emplaced within a vein to provide fluid communication between the reservoir and the vascular system of the patient.

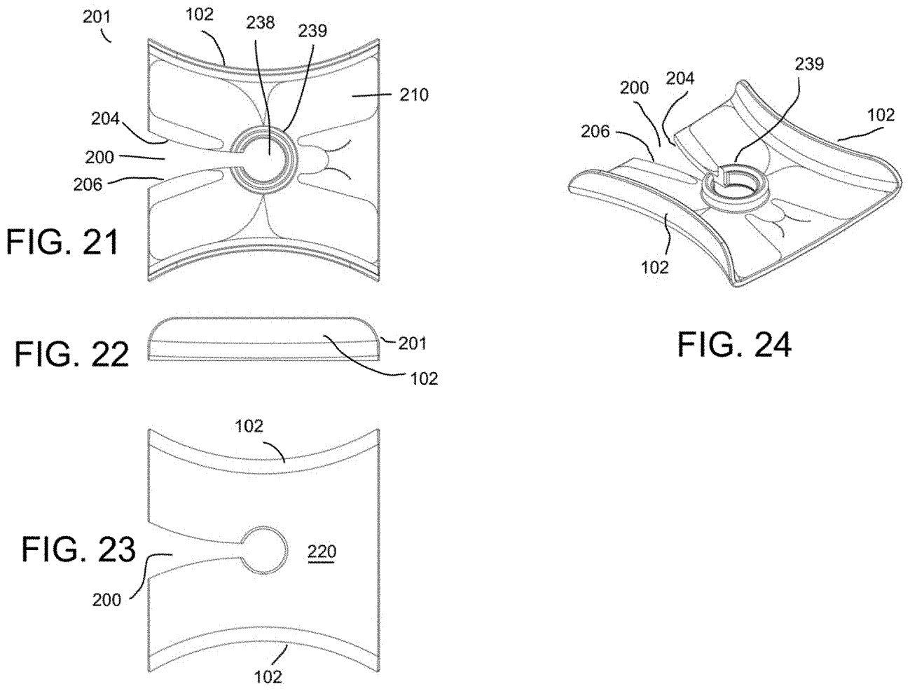

FIG. 3 shows a top perspective isolated view of locator tool 101 from FIG. 2. At least one magnet 160 may be provided along the rim of locator tool 101, such as a single ring magnet, or point magnets. Within port, preferably, one, two, or most preferably three pieces of MRM will be arrayed around a triangular shape embedded within the port as locator pins 142. Magnet 160 may be adapted to provide a magnetic field extending into the patient's body. Alternatively, magnet 160 may simply be a piece of magnetically responsive metal that does not create a significant magnetic field. First guide magnet 160 is preferably provided in tool 101 and is adapted to provide a magnetic field, and either interact with second lower magnet 158 (or MRM) or locator pins 142 (shown below), or mate with a magnetic field external to the patient's body. Locator tool 101 includes handles 102 on either side of the guide. Locator tool 101 also includes central aperture 138 through which a needle may be emplaced to access the port (as shown above). Locator tool 101 may include needle guide 136 with guide magnet 160, preferably in central aperture frame 139, which can provide magnetic field below the lower surface, or underside 116, of guide 136 and tool 101. In some embodiments, central aperture 138 is paired with channel 200 (shown in FIGS. 21-24 below), and needle guide 136 does not include a complete circumferential frame, but is surrounded less than three hundred-sixty degrees to provide for a temporary affixed tube (such as a drip as is known in the art) to be aligned along the skin and taped thereto without interfering with removal of needle guide.

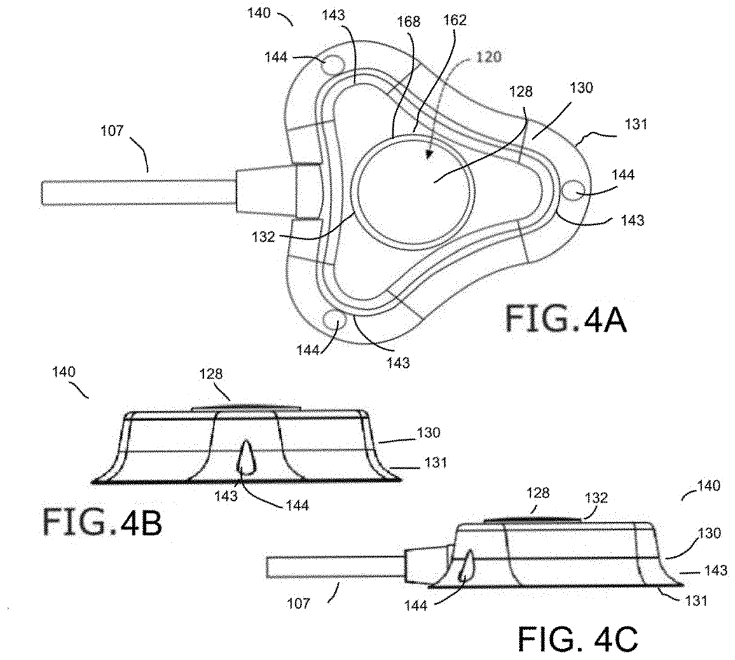

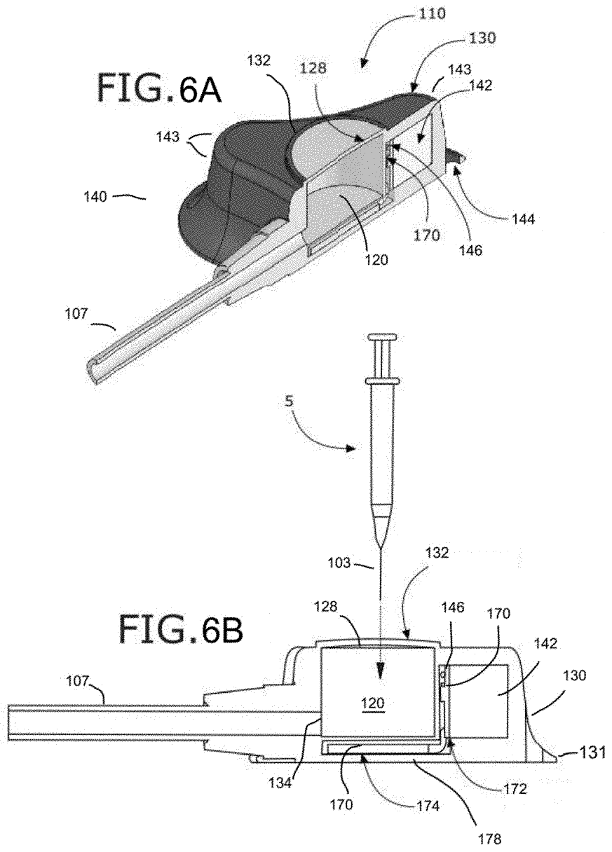

Referring now to FIGS. 4A-C and 5, a preferred embodiment of medical device includes a port 140 with housing 130. Port 140 includes septum 128 (preferably of compressed silicone) bound within the upper rim 132. Lower, or second, magnet or MRM 162 (as discussed below) may comprise a ring magnet 168, a ring of responsive metal, MRM, or preferably at least two points of magnetically responsive or magnetic material. In the embodiment as shown, port 140 includes three corners 143, each comer may be associated with a suture hole 144 to allow suturing into a client. Preferably, in this embodiment, port includes three MRM, each MRM associated with one of three comers 143 (as shown in cross-section in FIGS. 6A and 6B). Port 140 includes suture holes 144 to facilitate placement, and suturing into patient flesh below the skin. Suture holes also provide stability against movement relative body, which will be important due to the changing magnetic fields, and thus forces on MRM in device 110 and/or port 140. Port reservoir 120 is defined below septum 128. Cather 107 extends from housing 130 from catheter shield 119. Port 140 may include a generally triangular, or round, shape in the lower housing 131, such as flanged rim, to provide three suture holes 144 at each corner to provide for maximum stability of the implanted device. Preferably, rim 132 is circular, but may be oval or other shape known in the an. Alternative, or in addition, to tool magnet 160, housing may include locator pins 142 at each corner 143 with MRM. Locator pins interact with magnetic field created by magnet 160 in locator tool 101.

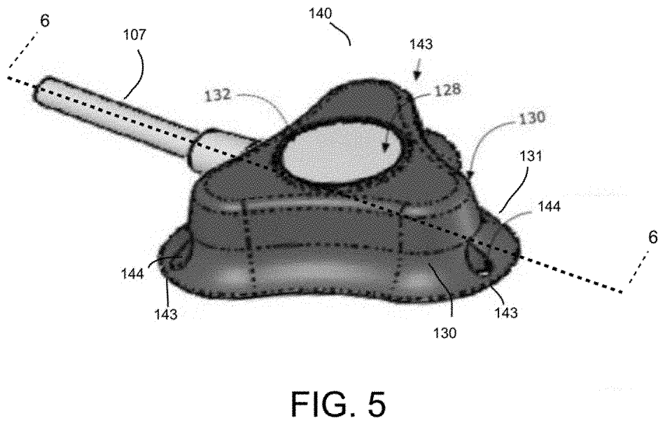

Referring to FIGS. 6A-6B, port 140 is shown in cross-sectional perspective and side views with needle/syringe shown off-scale (FIG. 6B). The medical device 110 may be beneficial for use by a user to increase reliability and ease of use of implantable medical port 140 for communicating liquids to and from a patient. As illustrated, the medical device comprises a medical port 140 for verifying (enabling) proper placement of a medical instrument 5 such as needle 103 with respect to the implantable medical device. The medical port 140 includes reservoir 120 adapted to store or provide access to fluid within an implantable housing 130 defining dimensions to the port 140 through which fluid access to the reservoir 120 is obtained. Housing 130 has upper rim 132 disposed towards the underside of a patient's skin (when implanted), aperture 134 (typically lower sidewall) adapted to provide fluid communication with a portion of the patient's vascular system (not shown). Housing may also include a flanged lower housing 131 to provide more stability and location for suturing. Septum 128 defines upper limit of reservoir 120. Housing 130 may also include locator pin 142 aside or next to the reservoir 120, preferably along corners 143 within housing 130. In the embodiment shown in FIGS. 4A-6B. port 140 includes three corners 143, and thus is preferred to include three locator pins 142. Locator pin is preferably comprised of MRM, possibly in a cylinder, block, or other shape to fit within housing. Port may also include a magnetic sensor (such as Reed switch or other) 146, to interact with a magnetic field, such as a magnet 160 in locator tool (not shown). When guide is placed over port, flexible printed circuit board (PCB) 172 may react to changes in the magnetic field to activate elements within the port, such as a light, chemical release, etc. In this embodiment, light 170 is a micro-LED along the sidewall. A lower PCB 174 may be included to active light, manage power, and/or support a further light emitter below reservoir 120. Flexible circuit board 172 and printed circuit board 174 may be electronically coupled. The light may be activated when the guide, such as a locator tool, is placed over the port, with the light penetrating the reservoir, septum, skin, and apparent through the central aperture of guide. A thermo-electric generator 178 may be coupled with one of the circuit boards, light emitter, and/or battery. In alternative versions, handheld tool may also include a power source that can initiate a light, or other feature, within port, as may be known in the art.

The medical device 110 may also include at least one light emitter 170 (LED or other such micro light or illuminating means) along at least one surface of the housing 130 (FIG. 6A-6B); the light emitter 170 may be adapted to emit light directly viewable through the skin of the patient such that the nurse or other practitioner is able to reliably locate the septum 128 on the port 140 with relative ease. In this way the present invention lends itself to efficient liquid drug administration and blood sampling. Using the medical device 110 permits that efficient communication of liquids to and from the patient be realized. Reservoir 120 is adapted for receiving medication from a percutaneously inserted hypodermic needle. As such, medical device 110 is designed and configured for verifying proper placement of a medical instrument 5 (FIG. 6B) with respect to an implantable medical device 112. In this particular embodiment of the medical system 100 the medical system 100 comprises: an implantable housing 130 defining a port 140 adapted to provide fluid communication to access a portion of the patient's vascular system.

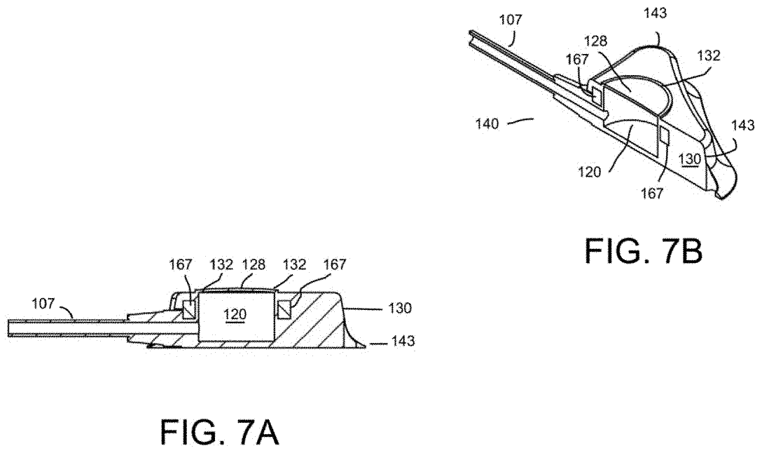

Referring to alternative embodiment of port 140 shown in cross section in FIGS. 7A and 7B, port is provided to allow fluid communication access to a vein, artery, or other portion of the patient's vascular system, preferably through catheter 107. Port 140 preferably includes housing 130 to surround reservoir 120, reservoir preferably located centrally within housing. Housing 130 may include corners 143. MRM of this embodiment may be in the shape of a ring, such as ring MRM 167. Ring MRM may be a ring magnet below rim 132, or slightly larger and within housing well below septum 128, as shown. Given the shape of the ring MRM, housing may not be required to have a shape with corners, but may rather include a more circular shape to accommodate ring MRM, without need for larger, or more numerous locator pins, preferably along corners 143 within housing 130.

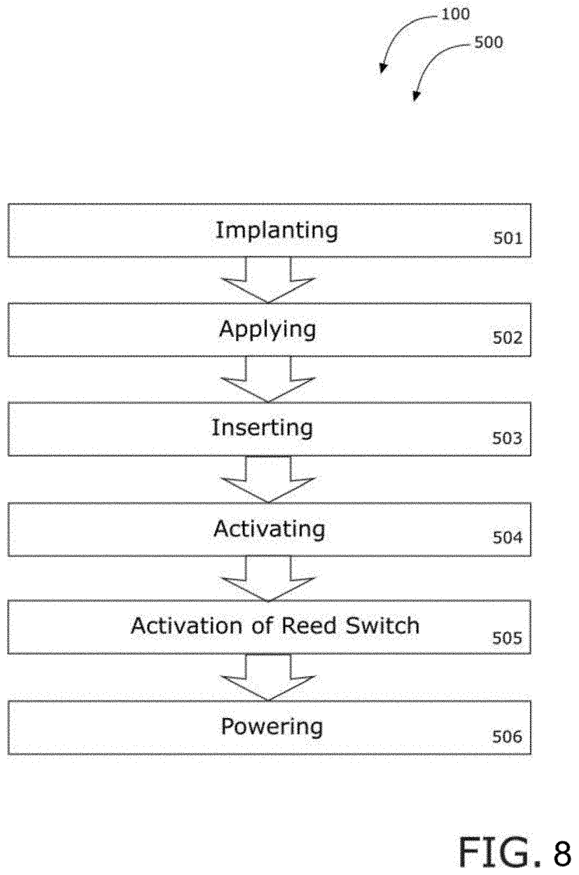

Referring now to FIG. 8 showing a flow diagram illustrating a method of use 500 for the medical system 100, according to an embodiment of the present disclosure. In particular, the method of use 500 may include one or more components or features of the medical system 100 as described above. As illustrated, the method of use 500 may include the steps of: step one 501, implanting a (provided) medical port under a film (otherwise the skin); step two 502, applying a MRM over the film to mate with an embedded magnet within the medical port; step three 503, inserting a hypodermic needle to supply medication into the medical port; step four 504, activating a light coupled to the medical port with the magnet; wherein the step of activating a light comprises activation of a Reed switch (step five 505), and step six 506 powering the light via a thermoelectric generator. Alternative or additional steps may be added as described above, or as commonly associated with port-related services as are known in the art.

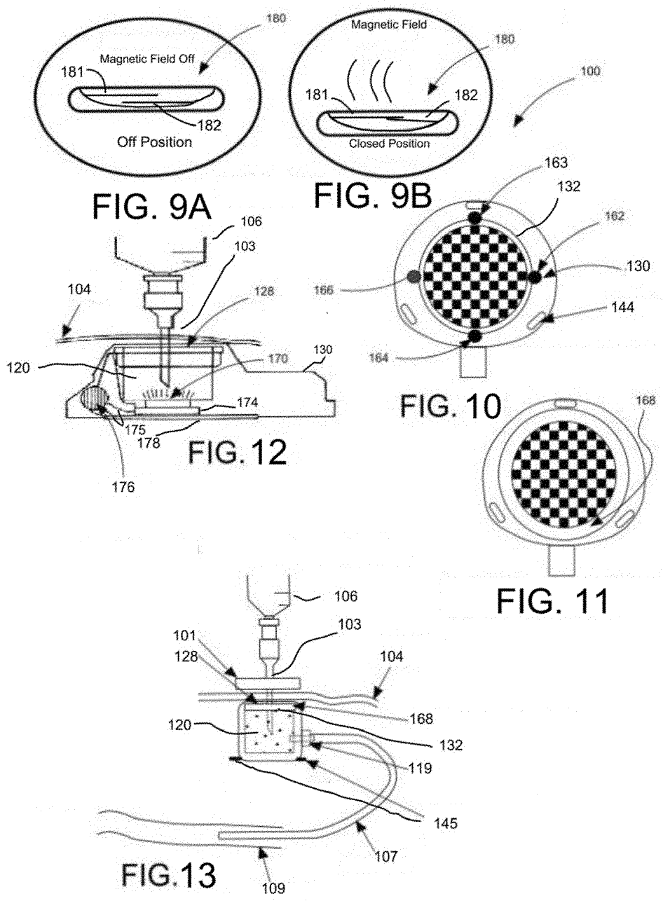

Referring to FIGS. 9A-9B, medical device may further comprise a Reed switch electrically coupled to the light emitter, and or electronics, and/or circuit board; Reed switch 180 adapted (configured) to be optionally connected via magnetic interaction with the MRM 160. Reed Switch 180 includes two terminals 181 and 182 of the medical port system in an OFF Position (magnetic field off) FIG. 9A when terminals 181 and 182 are apart, and a Reed Switch 180 in an ON Position (magnetic field on), FIG. 9B when terminals 181 and 182 in contact to close a circuit.

The housing 130 in certain embodiments comprises an upper rim 132; the upper rim 132 comprising a second lower magnet 162; and a handheld needle guide 136 comprising a central aperture 138. The housing 160 may further comprise an upper magnet 158, whereby the lower MRM 162 and the upper magnet 160 are adapted to magnetically mate. The medical system 100 may further comprise at least one light emitter 170 preferably coupled to the housing 130. The light emitter 170 is adapted for activation by a Reed switch 180; the Reed switch 180 being in magnetic communication with the upper magnet 160. When the upper magnet 160 and the lower magnet 162 are mated, an interior surface of the handheld needle guide 136 is circumscribed by at least one side wall of the implantable housing 130.

FIGS. 10-11 demonstrate a top view of alternative embodiments of the port 140 with septum 128 in view. Rim 132 may include a variety of MRM, preferably at least two, including MRM points 162, 163, 164, and 166. Each of MRM points 162, 163, 164, and 166 are here shown above and outside perimeter of rim 132. MRM points may be placed along the outer surface of the housing, or within the housing. Referring to FIG. 11, a single circumferential ring magnet 168 may be placed either over rim, or act as a rim. Alternatively, ring magnet, may simply be magnetizable, or magnetically responsive metals. Similarly, MRM points 162, 163, 164, and 166 may be of similar alternative substances, may be of arranged for alternating polarity, etc.

FIG. 12 shows needle 103 from syringe 106 passing through layer of skin 104 and being used to add or remove (communicate) fluid in reservoir 120. Needle 103 passes through the septum 128 into the reservoir 120. In some instances, a sensor may be emplaced in port 140 to detect breach of septum, breach of light within reservoir, or otherwise detect syringe, needle, etc. to activate light 170. Light here, is powered by battery 176. Battery may be coupled to light 170 through PCB 174 via wires 175. Battery 176 may be charged by thermo-electric generator 178 which may be placed below housing 130, or within housing as shown. Lower PCB 174 holds light emitter 170, such as an LED. Wires 179 are used to connect varied electronic components within the housing.

Referring now again to the at least one light emitter 170; the power source is electrically coupled to the at least one light emitter 170 (FIG. 12); wherein the power source may include power storage in a battery, capacitor, or other power storage device known in the art. The medical system 100 may further include a generator electrically coupled to the power source. The generator may comprise a thermoelectric generator or other suitable powering means. In this way the at least one light emitter 170 is able to be powered to provide illumination as needed/desired.

FIG. 13 shows a needle 103 communicating fluid through a locating tool as needle guide 136, through the skin 104 of a patient and piercing a septum 128 in series. Magnet ring 168 may be shown above septum to magnetically grip or interact with a magnet (previously 162) in needle guide 136. Port 140 may include catheter shield 119 to catheter 107 which may go on to enter blood vessel 109 (not shown to scale). Port 140 may be sutured to patient via suture plugs 145 in suture holes 144.

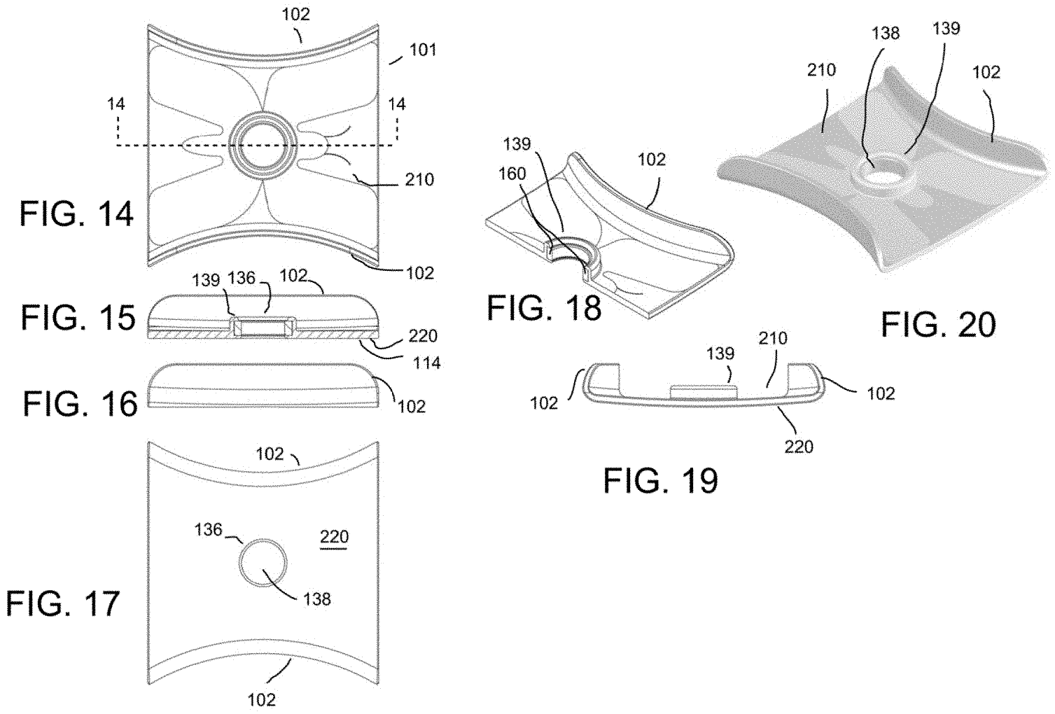

Referring now to an embodiment of locator tool 101 shown in FIGS. 14-20, locator tool 101, includes sides with handles 102 thereupon. Handle 102 are preferably bent up from the tool to provide ergonomic handling. Top side 210 is preferably flat and may include indicia 250. Indicia 250 is shown as a butterfly in FIGS. 13 and 19. Center of tool includes an aperture 138, preferably surrounded by a raised aperture frame 139. Aperture frame 139 may include magnet 160 or another first MRM. Together aperture 138 and aperture frame 139 provide for needle guide 136. Bottom side 220 may be outfitted with cover material 114. Cover 114 may include a soft material to provide comfort to user against skin.

Referring now to an alternative embodiment of locator tool 201 shown in FIGS. 21-24. locator tool 201, includes sides with handles 102 thereupon. Handle 102 are preferably bent up from the tool to provide ergonomic handling. Center of tool includes an aperture 238, preferably surrounded by a raised aperture frame 239. Raised aperture frame 239 only partially circumscribes aperture 238, and leaves a portion open, and may be C-shaped. Channel 200 is provided on one side of aperture 238. Channel 200 includes side walls 204 and 206 to define the width of channel. Channel 200 preferably emanates from central aperture e238 at an angle, preferably encompassing five to thirty degrees in angle. Aperture frame 139 may include magnet 160 or another first MRM. Channel allows fixing a connected tubing to the port outside of the body, such as a Huber needle, IV, etc.

I claim:

1. A medical system for verifying proper placement of a medical instrument with respect to an implantable medical device, said medical system comprising:
a. a reservoir adapted to store fluid;
b. an implantable housing defining a port through which fluid access to the reservoir is obtained; said housing comprising an upper rim disposed towards a body outer surface, and an aperture adapted to provide fluid communication with a portion of a patient's vascular system;
c. a locator tool comprising a first magnetically responsive material; and
d. said implantable housing comprising at least a second magnetically responsive material, said second magnetically responsive material adapted to respond to a magnetic field extending from, or responding to, the locator tool.

2. The medical system of claim 1 wherein said second magnetically responsive material is set along said upper rim.

3. The medical system of claim 2 wherein said second magnetically responsive material comprises a second ring magnet.

4. The medical system of claim 1 wherein said second magnetically responsive material is set within said housing.

5. The medical system of claim 1 wherein said housing further comprises at least a third magnetically responsive material.

6. The medical system of claim 5 wherein said housing further comprises at least a fourth magnetically responsive material

7. The medical system of claim 6 wherein said at least second, at least third, and at least fourth magnetically responsive materials are within the housing.

8. The medical system of claim 1 wherein said first magnetically responsive material comprises a ring magnet.

9. The medical system of claim 1 wherein said housing further comprises a septum bound within said upper rim.

10. The medical system of claim 1 further comprising at least one light emitter along at least one surface of said housing, said light emitter adapted to emit light directly viewable through skin of the patient.

11. The medical system of claim 10 further comprising a power source electrically coupled to said at least one light emitter.

12. The medical system of claim 11 further comprising a thermo-electric generator electrically coupled to said power source.

13. The medical system of claim 10 further comprising a Reed switch electrically coupled to said light emitter, said Reed switch adapted to be optionally connected via magnetic interaction with said at least second magnet.

14. A medical system for verifying proper placement of a medical instrument with respect to an implantable medical device, said medical system comprising:
a. an implantable housing defining a port adapted to provide fluid communication to access to a portion of the patient's vascular system; said housing comprising a lower magnetically responsive material; and
b. a handheld needle guide comprising a central aperture, and further comprising an upper magnetically responsive material, whereby said lower magnetically responsive material and upper magnetically responsive material adapted to magnetically male.

15. The medical system of claim 14 further comprising at least one light emitter coupled to said housing, said light emitter adapted for activation by a Reed switch, said Reed switch in magnetic communication with said upper magnet.

16. The medical system of claim 14 whereby when said upper and lower magnetically responsive material are mated, an interior surface of said handheld needle guide is circumscribed by at least one side wall of said implantable housing.

17. A method of supplying a medication to a patient comprising the steps of:
a. implanting a medical port under a film;
b. applying a locator tool magnetically responsive material over the film to mate with an embedded magnetically responsive material within the medical port; and
c. inserting a hypodermic needle to supply medication into, or draw fluid from, the medical port.

18. The method of claim 17 further comprising the step of activating a light coupled to the medical port via said step of applying.

19. The method of claim 18 wherein said step of activating a light comprises activation of a Reed switch.

20. The method of claim 18 further comprising the step of powering the light via a thermoelectric generator.
